(19) United States
(12) Patent Application Publication
(10) Pub. No.: US 2026/0320011 A1
(43) Pub. Date: Oct. 15, 2026

(54) DATASET ERROR IDENTIFICATION AND CORRECTION

(71) Applicant: Insight Direct USA, Inc., Chandler, AZ (US)

(72) Inventor: Michael Griffin, Wayland, MA (US)

(21) Appl. No.: 19/464,036

(22) Filed: Jan. 29, 2026

Related U.S. Application Data
(60) Provisional application No. 63/752,177, filed on Jan. 31, 2025.

(51) Int. Cl. G06F 16/215; G06F 16/22; G06N 3/045; G16H 10/60

(52) U.S. Cl. CPC G06F 16/215; G06F 16/2282; G06N 3/045; G16H 10/60

(57) ABSTRACT

A method of correcting database errors includes receiving tabular data, identifying a first error through analysis of the tabular data with an error-recognition model, retrieving a first error-correction function for correcting the first error type, and executing the first error-correction function to modify the tabular data to correct the first error. The first error belongs to a first error type of a plurality of error types and the error-correction function is retrieved from an error-correction function database.

### CROSS-REFERENCE TO RELATED APPLICATION

This application claims the benefit of U.S. Provisional Patent Application No. 63/752,177, filed Jan. 31, 2025, and entitled “DATASET ERROR IDENTIFICATION AND CORRECTION,” the disclosure of which is hereby incorporated by reference in its entirety.

### FIELD OF THE INVENTION

The present disclosure relates to tabular data, more particularly, systems and methods for automated identification and correction of errors in tabular data.

### BACKGROUND

Machine learning has a wide range of applications and various machine-learning models can be trained to make predictions useful for the performance of various tasks, such as classification, value prediction, content generation, etc. Some machine learning strategies use labeled data to train a machine-learning model to make predictions. Errors in labeled data are typically identified through manual review and label assignment by human operators, which can be time-intensive and labor-inefficient.

### SUMMARY

An example of a method of correcting database errors includes receiving tabular data, identifying a first error through analysis of the tabular data with an error-recognition model, retrieving a first error-correction function for correcting the first error type, and executing the first error-correction function to modify the tabular data to correct the first error. The first error belongs to a first error type of a plurality of error types and the error-correction function is retrieved from an error-correction function database.

An example of a system for correcting database errors includes an error-correction function database and a server communicatively connected to the error-correction function database. The server includes a processor and at least one memory encoded with instructions. The instructions, when executed, cause the processor to receive tabular data, identify a first error through analysis of the tabular data with an error-recognition model, retrieve a first error-correction function for correcting the first error type, and execute the first error-correction function to modify the tabular data to correct the first error. The first error belongs to a first error type of a plurality of error types and the error-correction function is retrieved from an error-correction function database.

The present summary is provided only by way of example, and not limitation. Other aspects of the present disclosure will be appreciated in view of the entirety of the present disclosure, including the entire text, claims, and accompanying figures.

### BRIEF DESCRIPTION OF THE DRAWINGS

FIG. 1 is a schematic diagram of an example of a system for identifying and correcting errors in tabular data.

FIG. 2 is a flow diagram of an example of a method of correcting errors in tabular data performable by the system of FIG. 1.

FIG. 3 is a flow diagram of a further example of a method of correcting errors in tabular data performable by the system of FIG. 1.

FIG. 4 is a flow diagram of yet a further example of a method of correcting errors in tabular data performable by the system of FIG. 1.

FIG. 5 is a flow diagram of an example of a method of training a machine-learning model using tabular data generated by the methods of any of FIGS. 2–4.

While the above-identified figures set forth one or more examples of the present disclosure, other examples are also contemplated, as noted in the discussion. In all cases, this disclosure presents the invention by way of representation and not limitation. It should be understood that numerous other modifications and examples can be devised by those skilled in the art, which fall within the scope and spirit of the principles of the invention. The figures may not be drawn to scale, and applications and examples of the present invention may include features and components not specifically shown in the drawings.

### DETAILED DESCRIPTION

The present disclosure relates to systems and methods for automated identification and correction of errors in tabular data. The systems and methods disclosed herein enable the automated correction of tabular data errors using a pre-generated library of correction functions and, further, enable the use of generative machine-learning models to generate new error-correction functions for error types that are not correctable by existing functions of the pre-generated library of correction functions. The error identification and correction functions described herein enable significant improvements in processing of raw, tabular data into labeled training data suitable for use for training a conventional machine-learning model, such as an artificial neural network. Notably, existing methods of error-correction of raw, tabular data require human intervention and, accordingly, are both labor- and time-intensive. Advantageously, the present disclosure provides methods that can be performed automatedly by a computer system, significantly reducing the time and labor required to remove errors from raw data before using that data for training a machine-learning model.

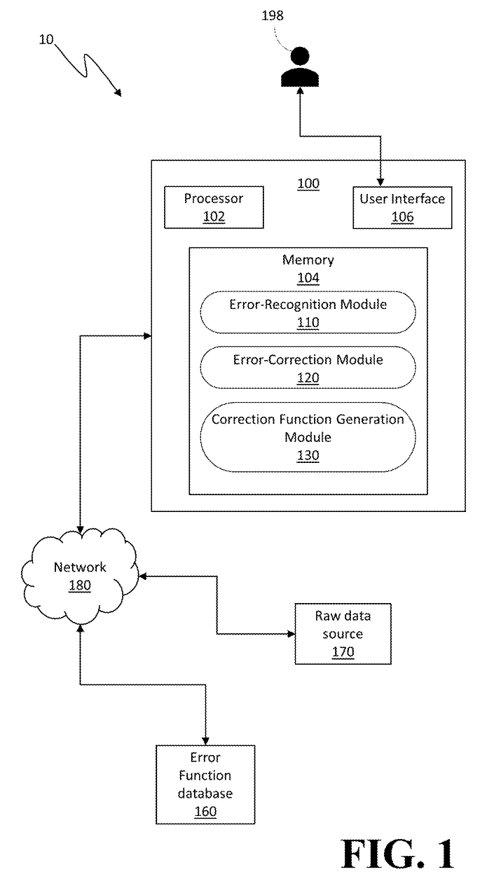

FIG. 1 is a schematic depiction of system 10, which is a system for automatedly identifying and correcting errors in tabular data. System 10 includes server 100, error-function database 160, raw data source 170, and network 180. Server 100 includes processor 102, memory 104, and user interface 106. Memory 104 stores error-recognition module 110, error-correction module 120, and correction function generation module 130. FIG. 1 also depicts user 198.

Server 100 is configured to automatedly identify and correct errors in tabular data. As will be explained in more detail subsequently, server 100 enables processing of data, including synthetic data, to remove database errors that can impeded, preclude, or otherwise impair training of a computer-implemented machine-learning model using the tabular data. The program(s) of server 100 enable the automated recognition of tabular errors and the retrieval of a correction function suitable for correcting the recognized error type.

Processor 102 can execute software, applications, and/or programs stored on memory 104. Examples of processor 102 can include one or more of a processor, a microprocessor, a controller, a digital signal processor (DSP), an application specific integrated circuit (ASIC), a field-programmable gate array (FPGA), or other equivalent discrete or integrated logic circuitry. Processor 102 can be entirely or partially mounted on one or more circuit boards.

Memory 104 is configured to store information and, in some examples, can be described as a computer-readable storage medium. Memory 104, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, memory 104 is a temporary memory. As used herein, a temporary memory refers to a memory having a primary purpose that is not long-term storage. Memory 104, in some examples, is described as volatile memory. As used herein, a volatile memory refers to a memory that that the memory does not maintain stored contents when power to the memory 104 is turned off. Examples of volatile memories can include random access memories (RAM), dynamic random access memories (DRAM), static random access memories (SRAM), and other forms of volatile memories. In some examples, the memory is used to store program instructions for execution by the processor. Memory 104, in one example, is used by software or applications running on server 100 (e.g., by a computer-implemented machine-learning model) to temporarily store information during program execution.

Memory 104 can further be configured for long-term storage of information. In some examples, memory 104 includes non-volatile storage elements. Examples of such non-volatile storage elements can include, for example, magnetic hard discs, optical discs, floppy discs, flash memories, or forms of electrically programmable memories (EPROM) or electrically erasable and programmable (EEPROM) memories.

User interface 106 is an input and/or output device and/or software interface, and enables an operator to control operation of and/or interact with software elements of server 100. For example, user interface 106 can be configured to receive inputs from an operator and/or provide outputs. User interface 106 can include one or more of a sound card, a video graphics card, a speaker, a display device (such as a liquid crystal display (LCD), a light emitting diode (LED) display, an organic light emitting diode (OLED) display, etc.), a touchscreen, a keyboard, a mouse, a joystick, or other type of device for facilitating input and/or output of information in a form understandable to users and/or machines.

Error-function database 160 is an electronic database that is connected to server 100 via network 180. Error-function database 160 stores error-correcting functions created by correction function generation module 130. Error function database 160 can be, for example, a structured database (e.g., a table or relational database), a semi-structured database (e.g., a hierarchical and/or nested database), and/or an unstructured database, but is generally configured to be queried by error-correction module 120 with an error type to retrieve an error-correction function for correcting that error type.

Raw data source 170 is an electronic data source and is or includes a data table suitable for error correction by the program(s) of server 100. Raw data source 170 is also connected to server 100 via network 100. Raw data source 170 can be or can be stored by or in, for example, a database. In some examples, the data table from raw data source 170 can be copied to memory 104 of server 100 prior to data entry error correction by the program(s) of server 100. Raw data source 170 can be of any suitable data type, can include any suitable number of records, and can include any suitable number of fields describing any suitable properties of the records. In some examples, raw data source 170 can be medical records, such as patient health records. Additionally and/or alternatively, raw data source 170 can be synthetic data (e.g., synthetic patient health records), such as synthetic data generated by repeated prompting of a computer-implemented machine-learning language model.

Error-function database 160 and/or raw data source 170 can optionally organize data using database management systems. As used herein, a “database management system” refers to a system of organizing data stored on a data storage medium. In some examples, a database management system described herein is configured to run operations on data stored on the data storage medium. The operations can be requested by a user and/or by another application, program, and/or software. A database management system can be implemented as one or more computer programs stored on at least one memory device and executed by at least one processor to organize and/or perform operations on stored data.

Network 180 is a network suitable for connecting and facilitating network communication between two or more of server 100, error-function database 150, and raw data source 170. Network 180 can include any suitable combination of local area network (LAN) and wide area network (WAN) elements or components to facilitate communication between two or more of server 100, error-function database 150, and raw data source 170. In some examples, network 180 can be or include the Internet.

Error-recognition module 110 is a software module of server 100 and includes one or more software programs for recognizing correctable errors in raw data (e.g., a data table) from raw data source 170. The program(s) of error-recognition module 110 are configured to automatedly recognize errors in data tables. Error recognition by the program(s) of error-recognition module 110 enables subsequent retrieval of the correct error-correction function from error function database 160. Error-recognition module 110 can include one or more program(s) for recognizing errors automatically. In some examples, error-recognition module 110 can include separate functions for recognizing each error type correctable by error-correction module 120. In yet further examples, error-recognition module 110 can include functions configured to or otherwise capable of recognizing more than one type of correctable error. In some of these examples, error-recognition module 110 can include a computer-implemented machine-learning model trained to recognize correctable errors in data table information.

In some examples, error-recognition module 110 can be configured to prompt a machine-learning language model to recognize errors in data table information. In at least some examples, error-recognition module 110 can be configured to prompt a machine-learning language model 110 to create an error-recognition function for a new type of error. Error-recognition module 110 can be configured to, for example, automatedly recognize new errors in a data table and cause the program(s) of correction function generation module 130 to generate a new error correction function for correcting the new error.

Error-correction module 120 is another software module of server 100 and includes one or more programs for retrieving error-correction functions from error function database 160 and executing those retrieved functions to correct errors in data table information. The program(s) of error-correction module 120 can be configured to receive an error identification from error-recognition module 110, and further to responsively retrieve an error-correction function from error function database 160 and execute that error-correction function to correct one or more errors in tabular data.

Correction function generation module 130 is another software module of server 100 and includes one or more programs for generating new error-correction functions and storing those new functions to error function database 160. Correction function generation module 130 generates new error-correction functions using a machine-learning language model. In particular, correction function generation module 130 is capable of automatedly generating prompts that prompt a machine-learning language model to generate a new error-correction function for a new error type (i.e., an error type that cannot be corrected using an existing function of error function database 160). Correction function generation module 130 then provides the prompt to the machine-learning language model and stores the output to error function database 160 as an error-correction function for correcting the new error type.

The modules of server 100 are shown as separate software components in FIG. 1 for explanatory clarity and convenience. In other examples, two or more of error-recognition module 110, error-correction module 120, and error function generation module 130 can be part of a single software program and/or application. Further, each of server 100, error function database 160, and raw data source 170 are depicted in FIG. 1 as single devices for explanatory convenience. In other examples, any of server 100, error function database, and raw data source 170 can be distributed across any suitable number of devices. Similarly, each of server 100, error function database 160, and raw data source 170 are depicted as single devices in FIG. 1 for explanatory convenience. In other examples, two or more of server 100, error function database 160, and raw data source 170 can be integrated into a single physical device and/or a single virtual device distributed across any suitable number of physical devices.

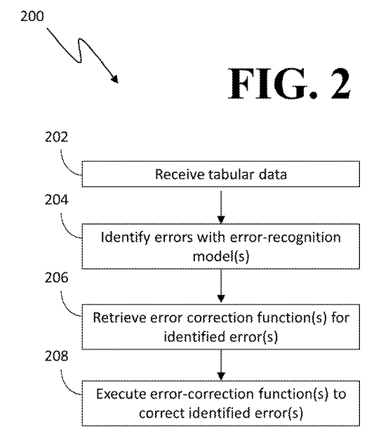

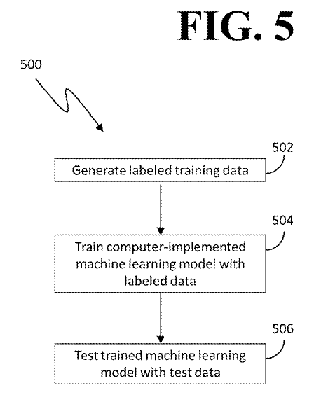

As will be explained subsequently and particularly with respect to the discussion of FIGS. 2–4, system 10 enables the automated identification and correction of errors in tabular data, and, in some examples, enables the automated generation of new functions (e.g., scripts) for correcting errors in tabular data. Tabular data corrected by system 10 can be used to train a computer-implemented machine-learning model via, e.g., method 500 (FIG. 5), discussed in more detail subsequently.

FIG. 2 is a flow diagram of method 200, which is a method of correcting errors in tabular data. Method 200 includes steps 202–208 receiving tabular data (step 202), identifying errors with one or more error recognition models (step 204), retrieving error correction functions for the identified error(s) (step 206), and executing the retrieved error correction function(s) to correct the identified error(s) (step 208). Method 200 is performable by system 10 (FIG. 1) and, accordingly, is discussed herein generally with reference to system 10. However, method 200 can more broadly be performed by any suitable computing system.

In step 202, server 100 receives tabular data. Tabular data can be provided via, e.g., user input by user 198 via user interface 106 and/or by retrieving tabular data from raw data source 170. The tabular data can include any suitable number of records and any suitable number of fields.

In step 204, the program(s) of error-recognition module 110 identify errors in the tabular data. Error-recognition module 110 is capable of detecting multiple types of errors in tabular data. Error-recognition module 110 can include a separate error-recognition function for each error type and/or can include error-recognition functions capable of recognizing multiple error types. In some examples, error-recognition module 110 can prompt a machine-learning language model to identify errors in the tabular data.

Errors recognized in step 204 render the tabular data unsuitable for training a machine-learning model, such as a neural network, to make predictions. For example, data entries can be unintentionally duplicated and the presence of two identical records can bias the training of the neural network. Additionally and/or alternatively, programs that automatedly assemble tabular data from, for example, text data can make errors that populate fields of the table with incorrect or otherwise improper values. For example, the same numerical or text data can appear twice in a single field. As yet an additional example, a field could be populated with an incorrect data type (e.g., natural-language text data in a data field for numeric values) or with a value outside of a range of anticipated values (e.g., a negative value for a field incapable of having or very unlikely to have negative values). Error-recognition module 110 can be configured to identify multiple error types likely to be present in tabular data and to store or otherwise generate a value or identifier for each identified error type.

In step 206, error-correction module 120 retrieves error-correction functions error function database 160 based on error identities provided by error-identification module 110 (i.e., in step 204). Error-correction module 120 uses the identity of each error type identified in step 204 to query error function database 160. In response to the queries, error function database 160 retrieves at least one function for correcting each queried error type.

In step 208, error-correction module 120 executes the error-correction function(s) retrieved in step 206 to correct errors recognized in step 204. Processor 102 can execute each error-correction function to modify data entries of the tabular data received in step 202.

Following step 208, the modified (i.e., error-corrected) tabular data is suitable for use in training a computer-implemented machine-learning model, such as a neural network. The modified tabular data can be stored to memory 104, to raw data source 170, and/or to any other suitable data storage device.

Method 200 advantageously allows system 10 and similar systems to automatedly correct recognized errors in tabular data using a library of stored error-correction functions (i.e., the error-correction functions stored to error function database 160).

Notably, the synthetic generation of training data is prone to introducing errors that render the resultant data unsuitable for subsequent training of a computer-implemented machine-learning model, such as an artificial neural network. Synthetic data is useful for training machine-learning models to demonstrate the ability of a machine-learning approach to solve a particular problem and/or to otherwise function as a proof-of-concept of a particular machine-learning approach without requiring time-consuming harvesting and processing of real data.

Furthermore, synthetic data can be particularly useful where real data is difficult to acquire real data otherwise required to validate and/or demonstrate applications of a machine-learning approach. As a specific example, patient health data can be difficult to acquire and often is legally-protected from disclosure to unauthorized parties. However, a third-party contractor may desire to propose a machine-learning approach to solve a gap in diagnosis, treatment, etc. to an entity legally- or otherwise-authorized to collect patient data. In these examples, synthetic patient data can be used instead of real patient data to train a machine-learning model for demonstration purposes as part of, for example, a project proposal.

In these examples, a generative machine-learning language model can be used to generate coherent patient data without requiring subject-matter expertise and, further, without requiring manual data creation for a dataset suitably large to train another machine-learning model to make predictions based on the patient health information. As a particular example, sequential, iterative prompting of a machine-learning model can be used to generate individual patient profiles, where each output is used by the machine-learning language model in combination with the following prompt. The written patient profile can then be transformed into tabular data. The machine-learning language model can be prompted to first create biographical information for a fictitious patient and then create basic patient health information for that fictitious patient, such as a quantity of encounters with the healthcare system and/or a description of whether those encounters were in-patient or out-patient visits. The machine-learning language model can then be prompted to generate data describing whether the fictitious patient’s vitals were measured during each visit and, if they were, to create values for those measurements. Based on those outputs, the machine-learning language model can then be prompted to generate a data describing whether any laboratory tests were performed during those fictitious visits and, if they were, to create data for those laboratory tests. The machine-learning language model can then be further prompted to create diagnoses based on the previously-generated data and to create medication or other treatment plans for those diagnoses. The resultant text data can be transformed into tabular data using any suitable software program. In at least some examples, a machine-learning model, such as a natural-language model and/or a generative language model can be used to extract text information and/or transform that extracted text information into tabular data.

The aforementioned sequential prompting method is merely one exemplary method of synthetic data generation using a generative machine-learning language model and, in other examples, a different method can be used to generate synthetic data.

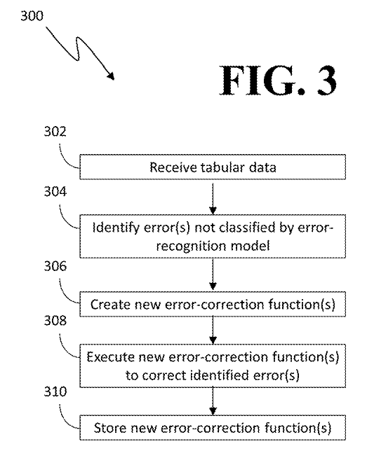

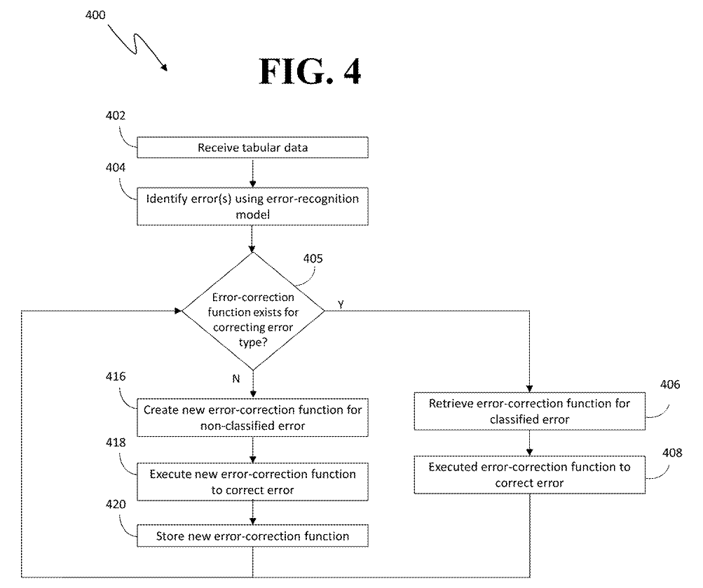

The aforementioned process of automatedly generating synthetic data by sequentially prompting a machine-learning language model and, subsequently, of automatedly or semi-automatedly converting that text data into a tabular format can cause various errors in the resultant tabular data, such as duplicate values or entries in a single cell. Method 200 is one example of a method of recognizing these types of errors. Method 300, discussed subsequently with respect to the description of FIG. 3, and method 400, discussed subsequently with respect to the description of FIG. 4, are two additional methods for recognizing and correcting errors in tabular data and can be used to correct errors derived from synthetic training data generation.

FIG. 3 is a flow diagram of method 300, which is another method of recognizing and correcting errors in tabular data. Method 300 includes steps 302–310 of receiving tabular data (step 302), identifying one or more errors not classified by the error-recognition model (step 304), creating new error-correction functions (step 306), executing the new error-correction function(s) to correct the identified error(s) (step 308), and storing the new error-correction function(s) (step 310). Method 300 is also performable by system 10 (FIG. 1) and is described herein with reference to FIG. 1 for explanatory convenience. More broadly, however, method 300 can be performed by any suitable computing system. Method 300 enables the identification of errors belonging to error types or categories that are not yet recognized by the program(s) and/or function(s) of error-recognition module 110.

In step 302, processor 102 receives tabular data. Step 302 is substantially similar to step 202 of method 200 (FIG. 2) and the description of step 202 of method 200 is applicable to step 302 of method 300. In some examples, the tabular data received in step 302 is the modified tabular data generated by step 208 of method 200 (FIG. 2).

In step 304, one or more errors not classified by an error-recognition model are identified. Method 300 can be performed following method 200, for example, to classify and correct errors belonging to error types that are not recognized by error-recognition module 110 and, therefore, were not captured by a prior iteration of method 200. Step 304 can be performed by a human operator and/or by the program(s) of server 100. For example, step 304 can be performed by prompting a machine-learning language model to identify errors in the modified tabular data produced by a previous iteration of method 200 (FIG. 2). The errors identified in step 304 can then be classified into categories of error for subsequent step 306.

In step 306, the program(s) of correction function generation module 130 generates a new error-correction function for correcting each category of error identified in step 304. Processor 102 can generate a new error-correction function by, for example, prompting a machine-learning language model to generate executable code (e.g., a script) that can be used to correct errors of the category of error. Processor 102 can also generate a prompt for the machine-learning language model to generate executable code (e.g., a script) that can be used by error-recognition module 110 to recognize the category of error.

In step 308, processor 102 executes the new function(s) generated in step 308 to correct the new errors identified in step 304. When executed, the new function(s) cause processor 102 to modify the data table to correct errors belonging to the identified error categories.

In step 310, processor 102 stores the new error-correction function(s) to error function database 160. Where a new function for recognizing the error category was also generated in step 306, processor 102 can modify error-recognition module 110 to include the new error-recognition function(s).

FIG. 4 is a flow diagram of method 400, which is another method of recognizing and correcting errors in tabular data. Method 400 includes steps 402–420 of receiving tabular data (step 402), identifying errors using an error-recognition model (step 404), determining whether an error-correction function exists for correcting the error type (step 405), retrieving the error-correction function for correcting a classified error (step 406), executing the error-correction function to correct error (step 408), creating a new error-correction function for a non-classified error (step 416), executing the new error-correction function to correct the error (step 418) and storing the new error-correction function (step 420). Method 400 is performable by system 10 (FIG. 1) and is described herein with reference to FIG. 1 for explanatory clarity and convenience. However, in other examples, method 400 can be performed by any suitable computing system.

Method 400 uses an agentic artificial intelligence model to identify errors and to both retrieve and execute error-correction functions. As such, in examples of system 10 capable of performing method 400, the functions of error-recognition module 110, error-correction module 120, and correction function generation module 130 are performed and/or executed by the agentic artificial intelligence model. As will be explained in more detail subsequently, method 400 advantageously enables the automated classification of both known and unknown error types, the automated retrieval of existing error-correction functions, and the automated generation of new error-correction functions.

In step 402, tabular data is received. Step 402 is substantially similar to step 202 of method 200 (FIG. 2), and the description of step 202 of method 200 herein is applicable to step 402 of method 400.

In step 404, the agentic artificial intelligence model identifies errors in the tabular data. The agentic artificial intelligence model can execute the error-recognition model of error-recognition module 110 to recognize errors in the tabular data. The agentic artificial intelligence model can also generate a prompt based on the tabular data as well as text representations of currently recognized error categories with instructions to a machine-learning language model to generate text representative of additional errors present in the tabular data.

In step 405, for an error type identified in step 404, the program(s) of server 100 determine whether an existing error-correction function exists for correcting the error type. If the error type is a known error type such that an error-correction function exists for correcting the error type, method 400 proceeds to steps 406–408. If the error type is a new error type or otherwise is one for which an error-correction function is not stored to error function database 160, method 400 proceeds to steps 416–420).

Steps 406 and 408 of method 400 are substantially similar to steps 206 and 208 of method 200 (FIG. 2), respectively, and the descriptions of steps 206 and 208 of method 200 are applicable to steps 406 and 408, respectively, of method 400.

Further, steps 416, 418, and 420 are substantially similar to steps 306, 308, and 310 of method 300 (FIG. 3), respectively. The descriptions of steps 306, 308, and 310 of method 300 are applicable to steps 416, 418, and 420, respectively.

After each error type is corrected, method 400 iterates back to step 405 to classify an additional error type and choose whether to follow steps 406–408 or steps 416-420 for correcting errors of the error type. When all error types have been corrected according to steps 406–408 or steps 416–420, method 400 can stop. Notably, while method 400 is described herein as iterating back to step 405 after completing one branch of error correction (i.e., steps 406–408 or steps 416–420), in other examples, two or more errors can be classified by step 405 and corrected using the appropriate set of steps 406–408 or steps 416–420 at the same time, at substantially the same time, or at least during a partially overlapping time period. In at least some examples, all error types identified in step 404 are processed simultaneously or substantially simultaneously via the subsequent steps of method 400, such that method 400 does not iterate.

FIG. 5 is a flow diagram of method 500, which is a method of training a machine-learning algorithm according to the present disclosure. Method 500 can be used to train machine-learning model based on data processed via any of method 200 (FIG. 2), method 300 (FIG. 3), and method 400 (FIG. 4). Method 500 includes steps of generating training data and test data (step 502), training a computer-implemented machine-learning model with the training data (step 504), and testing the trained computer-implemented machine-learning model with the test data (step 506). Method 500 can be used to train any suitable machine-learning model and can be used, for example, to train a neural network to score image focus.

In step 502, processor 102 generates labeled training data and labeled test data based on tabular data processed for error-correction via at least one of method 200 (FIG. 2), method 300 (FIG. 3), and method 400 (FIG. 4). Labeled data can be extracted from the tabular data to train the machine learning model to make a prediction based on one or more input values. In at least some examples, a portion (e.g., a majority) of the labeled data can be designated as training data and a remainder (e.g., a minority) of the labeled data can be designated as test data. In other examples, the error-corrected tabular data can be used as training data and additional data of the same kind can be used as test data.

In step 504, the training data is used to train the computer-implemented machine-learning model. As used herein, “training” a computer-implemented machine-learning model refers to any process by which parameters, hyperparameters, weights, and/or any other value related model accuracy are adjusted to improve the fit of the computer-implemented machine-learning model to the training data. Training in step 504 can be performed iteratively to iteratively adjust and improve the fit of the model to the training data.

In step 506, the trained computer-implemented machine-learning model is tested with test data. More specifically, a human operator or computer program can evaluate the performance of the machine-learning model by evaluating the fit of the model to the test data. The fit of the model can be evaluated to all or a portion of the test data generated or received in step 502.

In some examples, method 500 can be performed iteratively to improve the performance of the machine-learning model. More specifically, if the fit of the model to the test data determined in an iteration of step 506 is undesirable, step 504 can be repeated to further adjust the parameters, hyperparameters, weights, etc. of the model (e.g., via re-training) to improve the model. Step 506 can then be repeated with a new set of test data (e.g., a different subset of the test data generated in step 502) and/or the same set of test data to determine how the adjusted model fits the test data. If the fit continues to be undesirable, further iterations of steps 504 and 506 can be performed until the fit of the model becomes desirable.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of correcting database errors, the method comprising:
receiving, by a processor, tabular data;
identifying, by the processor, a first error through analysis of the tabular data with an error-recognition model, the first error belonging to a first error type of a plurality of error types;
retrieving, by the processor and from an error-correction function database, a first error-correction function for correcting the first error type; and
executing, by the processor, the first error-correction function to modify the tabular data to correct the first error.

2. The method of claim 1, and further comprising training a computer-implemented machine learning model using at least a portion of the modified tabular data.

3. The method of claim 1, wherein the error-recognition model is machine-learning model.

4. The method of claim 3, wherein the machine-learning model is a machine-learning language model.

5. The method of claim 4, wherein the machine-learning language model is a transformer model.

6. The method of claim 1, wherein the tabular data represents electronic patient health records.

7. The method of claim 6, wherein the electronic patient health records are synthetic patient health records, and further comprising:
generating each electronic patient health record by sequentially prompting a machine-learning language model with each prompt of a set of synthetic health record generation prompts; and
generating the tabular data by extracting text data from the synthetic patient health records.

8. The method of claim 1, and further comprising:
identifying a second error belonging to a second error type;
determining that the error-correction function database does not contain a function for correcting the second error type;
in response to determining that the error-correction function database does not contain a function for correcting the second error type, generating a second-error correction function for correcting the second error type;
storing the second error-correction function to the error-correction function database; and
executing, by the processor, the second error-correction function to modify the tabular data to correct the second error.

9. The method of claim 8, wherein generating the second-error correction function comprises prompting a machine-learning language model to generate the second error-correction function for correcting the second error type.

10. The method of claim 9, and further comprising updating the error-recognition model to identify errors belonging to the second error type.

11. The method of claim 1, wherein:
analyzing the tabular data using the error-recognition model to identify the first error comprises generating an identity of the first error type using the error-recognition model; and
retrieving the first error-correction function comprises querying the error-correction function database with the identity of the first error type generated by the error-recognition model.

12. The method of claim 1, and further comprising:
identifying, by the processor, a second error through analysis of the tabular data with the error-recognition model, the first error belonging to a second error type of a plurality of error types;
retrieving, by the processor and from an error-correction function database, a second error-correction function for correcting the second error type; and
executing, by the processor, the second error-correction function to modify the tabular data to correct the second error.

13. The method of claim 12, and further comprising:
identifying, by the processor, a third error through analysis of the tabular data with the error-recognition model, the third error belonging to the first error type of the plurality of error types;
retrieving, by the processor and from an error-correction function database, the first error-correction function for correcting the first error type; and
executing, by the processor, the first error-correction function to modify the tabular data to correct the third error.

14. A system for correcting database errors, the system comprising:
an error-correction function database; and
a server communicatively connected to the error-correction function database, the server comprising:
a processor; and
at least one memory encoded with instructions that, when executed, cause the processor to:
receive tabular data;
identify a first error through analysis of the tabular data with an error-recognition model, the first error belonging to a first error type of a plurality of error types;
retrieve, from the error-correction function database, a first error-correction function for correcting the first error type; and
execute the first error-correction function to modify the tabular data to correct the first error.

15. The system of claim 14, wherein the instructions, when executed, cause the processor to train a computer-implemented machine learning model using at least a portion of the modified tabular data.

16. The system of claim 15, wherein the error-recognition model is machine-learning model.

17. The system of claim 14, wherein the tabular data represents electronic patient health records.

18. The system of claim 17, wherein the electronic patient health records are synthetic patient health records, and wherein the instructions, when executed, further cause the processor to:
generate each electronic patient health record by sequentially prompting a machine-learning language model with each prompt of a set of synthetic health record generation prompts; and
generate the tabular data by extracting text data from the synthetic patient health records.

19. The system of claim 14, wherein the instructions, when executed, further cause the processor to:
identify a second error belonging to a second error type;
determine that the error-correction function database does not contain a function for correcting the second error type;
generate a second-error correction function for correcting the second error type in response to determining that the error-correction function database does not contain a function for correcting the second error type;
store the second error-correction function to the error-correction function database; and
execute the second error-correction function to modify the tabular data to correct the second error.

20. The system of claim 19, wherein the instructions, when executed, cause the processor to generate the second-error correction function by prompting a machine-learning language model to generate the second error-correction function.
